(19) United States
(12) Patent Application Publication
(10) Pub. No.: US 2006/0254594 A1
(43) Pub. Date: Nov. 16, 2006

(54) Laryngeal mask airway

(76) Inventors: Edward Gologorsky, Miami Beach, FL (US); Angela Gologorsky, Miami Beach, FL (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/127,427

(22) Filed: May 12, 2005

(51) Int. Cl. A62B 9/06; A61M 16/00

(52) U.S. Cl. 128/207.14; 128/207.15; 128/207.16; 128/200.26

(57) ABSTRACT

A laryngeal mask airway includes a shaft having a lumen. A mask at the distal end of the shaft is dimensioned to be received within the supraglottic pharynx above the vocal cords of a patient. A balloon is located on the periphery of the mask and is inflatable to form a seal between the mask and the walls of a surrounding lumen. In a first aspect of the invention, the device includes a means for detachably coupling the mask to the distal end of the shaft such that the lumen of the shaft is in communication with an aperture in the mask, and such that when the shaft is detached from the mask, the distal end of the shaft can be advanced through the aperture and beyond the mask. In a further aspect of the invention, a method is disclosed of using a mask of the type just described to intubate a patient. In another aspect of the invention, the device includes a specially configured balloon which, when inflated, is larger on its posterior, superior, and lateral sides and thinner on an anterior side. In still another embodiment, longitudinal lines of weakness are formed on the shaft of the laryngeal mask airway. The shaft can be split along these lines of weakness, thereby making the laryngeal mask airway peelable.

### TECHNICAL FIELD

The present invention relates generally to devices and methods for intubating patients, and relates more specifically to an improved laryngeal mask airway.

### BACKGROUND OF THE INVENTION

“Intubation” means placing an endotracheal tube (ETT) in the trachea to permit positive pressure ventilation of lungs. This tube usually consists of a plastic shaft with an inflatable balloon in the end. This balloon is inflated after the intubation to seal the airway and to prevent a possible aspiration of gastric contents. Visualization with a laryngoscope is the standard procedure.

However, because of variable anatomy, sometimes the intubation is difficult, and other means of airway maintenance are required. Most definitive is fiberoptic (FOB) intubation, using a flexible fiberoptic scope as a guide to enter the trachea. Thereafter an ETT is advanced over the FOB.

In the last decade a special device, a laryngeal mask airway (LMA) has gained popularity. Basically, a LMA is a shaft with a spoon-like balloon in the end. It is placed in the supraglottic pharynx above the vocal cords, and the inflated balloon serves to seal the pharynx to allow positive pressure ventilation. It is a very versatile device, but it is not a substitute for an ETT, for the following reasons: first, it does not protect lungs from aspiration; second, its prolonged use may be contraindicated in some patients, and third, many procedures cannot be done without an ETT.

Among other things, LMA can be used as a conduit for ETT placement in situations of difficult laryngoscopy and unsuccessful intubation, and makes the FOB tracheal positioning easier since it lifts away the soft tissues and provides a direct unimpeded view of the vocal cords. The procedure is somewhat cumbersome, and consists of several steps. The LMA is first placed. Then, a fiberoptic bronchoscope with a smaller diameter ETT is advanced through the LMA into the trachea. Because of almost similar length of the ETT and LMA, the ETT will enter only 1-2 cm below the vocal cords. Finally the ETT balloon is inflated, the LMA balloon is deflated, and both the LMA and ETT remain in the airway.

There are practical problems associated with this approach. First, both devices are left in the airway. Further, the relatively shallow position of the ETT in the trachea presents the potential of easy dislodgement. Additionally, only the small diameter of the ETT can be advanced through the LMA.

Some of the problems have been addressed by modification of an LMA, called an intubating LMA, which features a relatively short shaft bent at a 90 degrees to the handle, allowing somewhat easier advancement of the ETT. However, this change of design creates a new set of difficulties, most prominent being a short shaft, making the placement of the LMA difficult, and impossible in some cases of difficult airways, particularly those with a long distance from the mouth to the pharynx.

### SUMMARY OF THE INVENTION

Stated generally, the present invention comprises a laryngeal mask airway including a shaft having a lumen. A mask at the distal end of the shaft is dimensioned to be received within the supraglottic pharynx above the vocal cords of a patient. A balloon is located on the periphery of said mask and is inflatable to form a seal between the mask and the walls of a surrounding lumen. In a first aspect of the invention, the device includes a means for detachably coupling the mask to the distal end of the shaft such that the lumen of the shaft is in communication with an aperture in the mask, and such that when the shaft is detached from the mask, the distal end of the shaft can be advanced through the aperture and beyond the mask.

In another aspect of the invention, the device includes a specially configured balloon which, when inflated, is larger on its anterior and lateral sides and thinner on its posterior side. This balloon configuration allows supraglottic or infraglottic ventilation and avoids cumbersome, and potentially dangerous, manipulation of the patient's airway in emergent situations

In a further aspect of the invention, a method of intubating a patient comprises the following steps. The distal end of a laryngeal mask airway comprising a shaft and a mask at the distal end of the shaft is inserted into the mouth of a patient. The distal end of the laryngeal mask airway is advanced until the mask is seated within the supraglottic pharynx above the vocal cords of the patient. A balloon on the periphery of the mask is inflated to form a seal between the mask and the adjacent tissue. The distal end of the shaft is uncoupled from the mask, and the distal end of the shaft is advanced beyond the mask and into the trachea of the patient.

Objects, features, and advantages of the present invention will become apparent upon reading the following specification, when taken in conjunction with the drawings and the appended claims.

### BRIEF DESCRIPTION OF THE DRAWINGS

FIG. 1 is a side view of a mask of a laryngeal mask airway according to a disclosed embodiment of the invention.

FIG. 2 is a top view of the mask of FIG. 1 with a shaft coupled thereto.

FIG. 3 is a cross-sectional view taken along line 3-3 of FIG. 2.

FIG. 4 is a cross-sectional view taken along line 4-4 of FIG. 2.

FIG. 5 is a cross-sectional view taken along line 5-5 of FIG. 2.

FIG. 6 is a side view of an alternate embodiment of a mask showing the insertion of a shaft.

FIG. 7 is a side view of the mask and shaft of FIG. 6 showing the shaft coupled to the mask to comprise a laryngeal mask airway.

FIG. 8 is a schematic view showing the laryngeal mask airway of FIG. 7 positioned within a patient.

FIG. 9 is a schematic view of the laryngeal mask airway and patient of FIG. 8 showing the shaft uncoupled from the mask and advanced for use as an endotracheal tube.

### DETAILED DESCRIPTION OF THE DISCLOSED EMBODIMENT

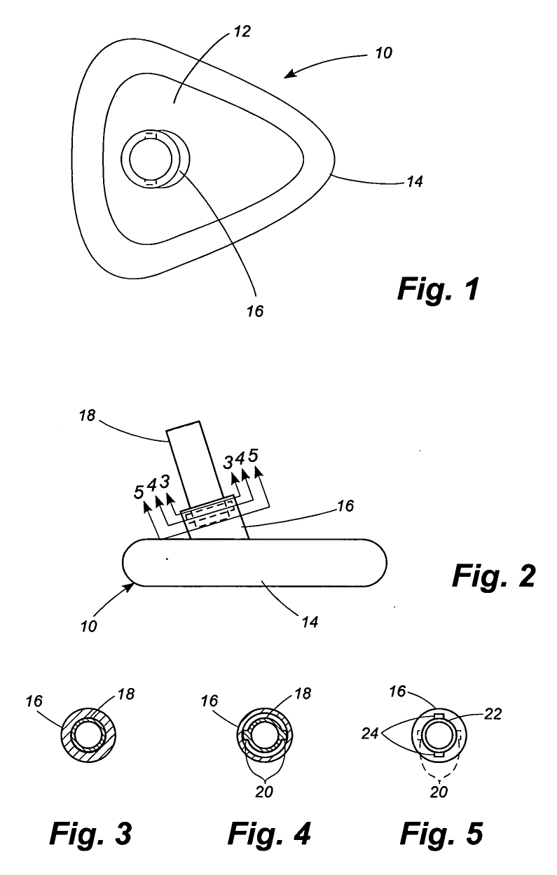

Referring now to the drawings, in which like numerals indicate like elements throughout the several views, FIGS. 1-5 illustrate a mask 10 of a laryngeal mask airway according to a disclosed embodiment of the invention. The mask 10 includes a body portion 12, an inflatable balloon 14 around its periphery, and a coupler 16 for coupling a shaft 18 to the mask.

The shaft 18 includes lobes 20 (FIG. 4) adjacent the distal end 22 of the shaft which engage the coupler 16. The coupler has corresponding slots 24 (FIG. 5) configured to receive the lobes 20 of the shaft 18 therethrough. Once the lobes 20 clear the slots 24, the shaft 18 can be rotated out of alignment with the slots 24 as shown in FIG. 5, coupling the shaft to the mask 10. Conversely, the shaft 18 can be rotated to re-align the lobes 20 with the slots 24, permitting the distal end 22 of the shaft 20 to be advanced beyond the mask 10.

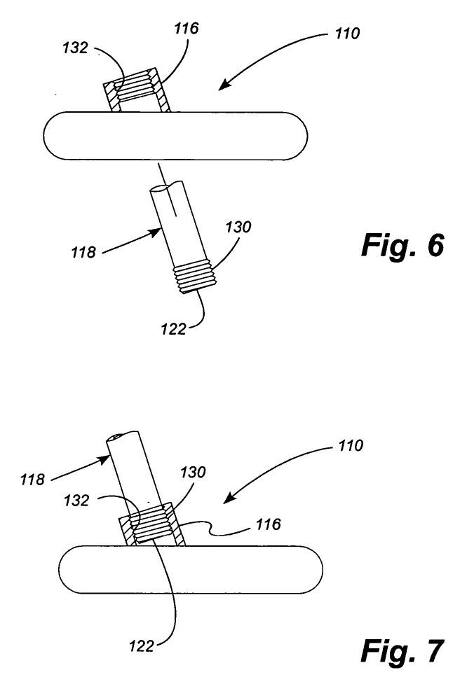

FIGS. 6 and 7 illustrate an alternate embodiment of a mask 110 and shaft 118. Rather than the shaft coupling to the mask by way of lobes and cooperating slots, as in the mask 10 and shaft 18, the mask 110 and shaft 118 include a threaded coupling arrangement. The periphery of the shaft 118 adjacent the distal end 122 includes male threads 130, and the coupler 116 of the mask 110 includes cooperating female threads 132. When the threads 130, 132 are engaged, as shown in FIG. 7, the mask 110 and shaft 118 are coupled. By turning the shaft 118 to disengage the threads 130, 132, the distal end 122 of the shaft can be advanced beyond the mask 110.

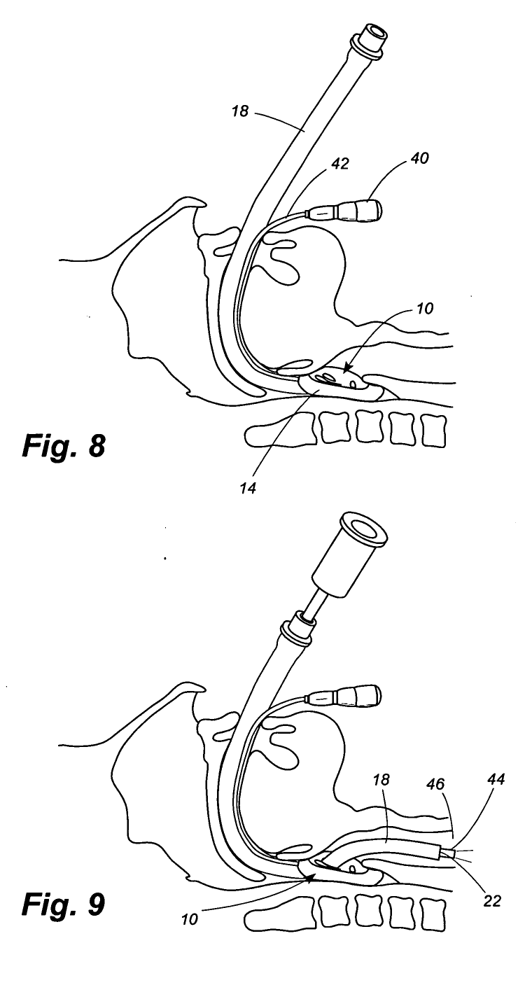

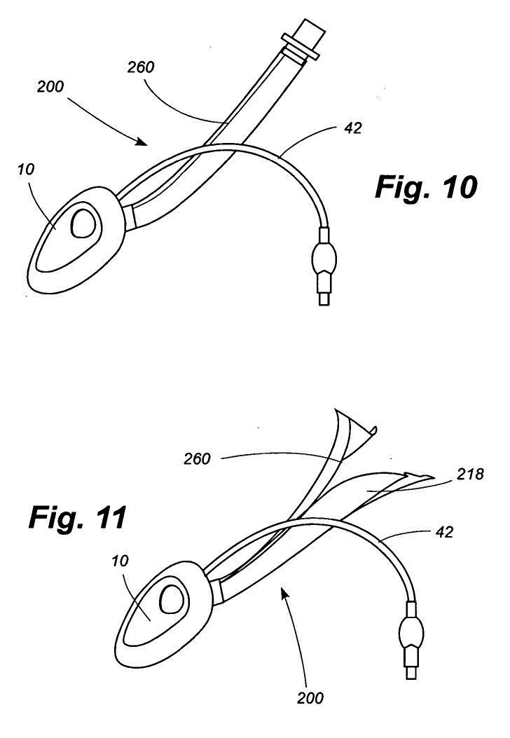

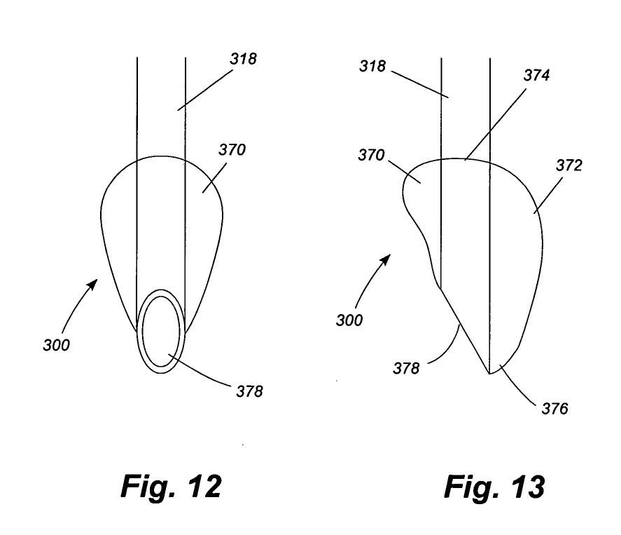

FIGS. 8 and 9 illustrate the operation of the mask 10. Referring first to FIG. 8, the mask 10 and shaft 18 are initially coupled. The mask 10 at the end of the shaft 18 is advanced to a location within the supraglottic pharynx above the vocal cords. The balloon 14 around the periphery of the mask 10 is inflated using inflation valve 40 and inflation line 42 in the conventional manner to seal the pharynx to allow positive pressure ventilation.

With reference now to FIG. 9, the shaft 18 has been rotated with respect to the mask 10 to uncouple the shaft from the mask. The distal end 22 of the shaft is advanced beyond the mask 10 and into the trachea 44 of the patient under guidance of a flexible fiberoptic scope 46. After the successful advancement of the shaft into tracheal position, the mask 10 is deflated and removed from supraglottic position by pulling on the inflation line 42.

FIGS. 10 and 11 illustrate another possible modification of a laryngeal mask airway 200. Longitudinal lines of weakness 260, circumferentially spaced 180° apart, are formed on the shaft 218 of the LMA. The shaft 218 of the LMA 200 can be split along these lines of weakness 260, thereby making the LMA peelable. That is, the sides of the LMA shaft 218 can be peeled apart, as a FOB, loaded with the standard ETT, is advanced down the shaft of LMA in the trachea. Having placed the LMA, the physician places the FOB, loaded with ETT, into the trachea, using the LMA as a guide. The ETT is slid over FOB, and correct placement is confirmed by FOB. Then the shaft is peeled apart and the LMA is removed in its entirety. The peels of shaft 218 and the inflation line 42 can be used to remove the LMA spoon-like mask 10 from the pharynx once the ETT is in the trachea. This modification allows the one-step removal of the LMA 200 after intubation, significantly decreasing the possibility of ETT dislodgement, soft tissue injury, or both.

A third embodiment of a laryngeal mask airway 300 is illustrated in FIGS. 12 and 13. For convenience of description, all of the directions (anterior, posterior, superior, inferior) as used herein are given in reference to the patient in a horizontal position, with the anesthesiologist standing at the patient's head, facing the patient. The anesthesiologist's right is to the patient's right, left to left. “Posterior” is the direction towards the floor, “anterior” towards the ceiling, “superior” towards the patient's head, and “inferior” towards the patient's toes.

The laryngeal mask airway 300 includes a thin-walled, asymmetrical balloon 314 mounted at the end of a shaft 318. The balloon includes an anterior portion 370, a posterior portion 372, a superior portion 374, and an inferior portion 376. When the balloon 314 is inflated, it inflates primarily in the posterior and superior directions. The shaft has an opening 378.

The laryngeal mask airway 300 functions as follows. Inflating mostly posteriorly, the balloon moves the tube's opening anteriorly, bringing it closer to the laryngeal inlet (since the laryngeal inlet is situated anteriorly to the esophageal opening), and thus vastly improving chances of both good ventilation (when used in a manner similar to LMA), and of glottic visualization by FOB. The superior part of the balloon provides the seal, allowing for the ventilation. The superior part of the balloon is mostly symmetrical, like a torus, with the shaft in the middle. It is contiguous with the posterior part, which is like a ladle, surrounding the shaft from behind, and gradually tapering laterally bilaterally, almost disappearing anteriorly. Essentially, it is an ETT with a thin-walled collapsible high volume asymmetrical balloon. In a supraglottic position it requires high volume (probably close to 30-40 cc) to push the bevel of the shaft to the laryngeal inlet, and then will need to be able to be completely collapsible (by removing the air), and will be reinflated in the trachea once the shaft is advances past the vocal cords, with a smaller volume (guided by the air leak or by the pressure in the pilot valve).

It will be understood that all of the embodiments described above may come in various appropriate pediatric and adult sizes.

Finally, it will be understood that the preferred embodiment has been disclosed by way of example, and that other modifications may occur to those skilled in the art without departing from the scope and spirit of the appended claims.

What is claimed is:

1. In a laryngeal mask airway comprising a shaft having a lumen and having proximal and distal ends, a mask at the distai end of said shaft dimensioned to be received within the supraglottic pharynx above the vocal cords of a patient, said mask having an aperture therein, and a balloon located on the periphery of said mask and inflatable to form a seal between said mask and the walls of a surrounding lumen, the improvement comprising:
means for detachably coupling said mask to said distal end of said shaft such that said lumen of said shaft is in communication with said aperture, and such that when said shaft is detached from said mask, said distal end of said shaft can be advanced through said aperture and beyond said mask.

2. The laryngeal mask airway of claim 1, wherein said means for detachably coupling said mask to said distal end of said shaft comprises mating threads on said mask and said distal end of said shaft.

3. The laryngeal mask airway of claim 1, wherein said mask further comprises a slot in communication with said aperture, and wherein said means for detachably coupling said mask to said distal end of said shaft comprises a lug formed on said shaft dimensioned to be received through said slot such that when said lug is inserted through said slot and said shaft is rotated, said shaft is coupled to said mask.

4. The laryngeal mask airway of claim 1, further comprising first and second lines attached at one end to said mask, each of said lines being of sufficient length to extend to a location outside of said patient when said mask is positioned within the supraglottic pharynx of said patient, whereby said mask can be extracted from said patient by pulling on said lines.

5. The laryngeal mask airway of claim 1, wherein one of said lines comprises an inflation lumen for inflating said balloon on said periphery of said mask.

6. The laryngeal mask airway of claim 5, wherein said one of said lines comprising said inflation lumen further comprises an inflation port on a free end thereof.

7. The laryngeal mask airway of claim 1, wherein said shaft further comprises at least one longitudinally extending line of weakness formed on said shaft, whereby said shaft can be separated along said line of weakness to detach said shaft from said mask.

8. The laryngeal mask airway of claim 7, wherein said line of weakness comprises a scored line.

9. The laryngeal mask airway of claim 7, wherein said line of weakness comprises perforations.

10. In a laryngeal mask airway comprising a shaft having a lumen and having proximal and distal ends, a balloon at the distai end of said shaft dimensioned to be received within the supraglottic pharynx above the vocal cords of a patient, said balloon having proximal, distal, superior, and anterior aspects, and said balloon inflatable to form a seal between said distal end of said shaft and the walls of a surrounding lumen, the improvement comprising:
said balloon being configured to inflate primarily in the superior and posterior directions.

11. The laryngeal mask airway of claim 10, wherein said superior aspect of said balloon is shaped like a torus.

12. A method for intubating a patient, comprising the steps of:
inserting into the mouth of a patient the distal end of a laryngeal mask airway comprising a shaft and a mask at the distal end of said shaft;
advancing said distal end of said laryngeal mask airway until said mask is seated within the supraglottic pharynx above the vocal cords of said patient;
inflating a balloon on the periphery of said mask to form a seal between said mask and the adjacent tissue;
uncoupling said distal end of said shaft from said mask; and
advancing said distal end of said shaft beyond said mask and into the trachea of said patient.

13. The method of claim 12, further comprising the step, prior to said step of advancing said distal end of said shaft beyond said mask and into the trachea of said patient, of introducing a fiberoptic tube into said shaft and advancing a forward end of said fiberoptic tube to a point at the forward end of said shaft; and
wherein said step of advancing said distal end of said shaft beyond said mask and into the trachea of said patient is performed by visualizing said trachea of said patient by way of said fiberoptic tube.

14. The method of claim 12, further comprising the step, after said distal end of said shaft has been advanced into said trachea, of removing said mask from said patient over said shaft while maintaining said shaft in place.

15. The method of claim 12, wherein said step of uncoupling said distal end of said shaft from said mask comprises the step of rotating said shaft with respect to said mask.

16. A method for intubating a patient, comprising the steps of:
advancing the distal end of a shaft into the supraglottic pharynx above the vocal cords of said patient, said shaft having an opening in said distal end and an inflatable balloon operatively associated with said distal end;
inflating said balloon in a posterior direction so as to move said opening of said shaft in an anterior direction and bringing it closer to the laryngeal inlet; and
inflating said balloon in a superior direction so as to create a seal between the balloon and the walls of the pharynx.
